(19) United States
(12) Patent Application Publication
(10) Pub. No.: US 2023/0022749 A1
(43) Pub. Date: Jan. 26, 2023

(54) VITAL SIGN MEASUREMENT DEVICE

(71) Applicant: SUBARU CORPORATION, Tokyo (JP)

(72) Inventor: Isamu NAGASAWA, Tokyo (JP)

(21) Appl. No.: 17/858,505

(22) Filed: Jul. 6, 2022

(30) Foreign Application Priority Data
Jul. 19, 2021 (JP) 2021-119191

(51) Int. Cl. A61B 5/0205; A61B 5/00; A61B 5/11

(52) U.S. Cl. CPC A61B 5/02055; A61B 5/6893; A61B 5/1116; A61B 5/6826

(57) ABSTRACT

A vital sign measurement device includes at least one sensor that is provided at a seat, and that is configured to contact a finger of a measurement subject and to measure a vital sign of the measurement subject. The seat includes a seat surface portion on which a femoral region and buttocks of the measurement subject are to be placed and a seat back capable of being disposed along a back of the measurement subject. The at least one sensor includes a side-portion sensor that is disposed below a side edge of the seat surface portion.

### CROSS-REFERENCE TO RELATED APPLICATIONS

The present application claims priority from Japanese Patent Application No. 2021-119191 filed on Jul. 19, 2021, the entire contents of which are hereby incorporated by reference.

### BACKGROUND

The disclosure relates to a vital sign measurement device that measures a vital sign of a measurement subject seated on a seat.

Detecting a vital sign (biological information) of an occupant, such as a driver, in a vehicle, such as an automobile, is proposed.

As a technology related to measurement of biological information of an occupant in a vehicle, for example, Japanese Unexamined Patent Application Publication No. 2016-190582 describes a technology in which a vital sign, such as breathing information or heartbeat information, is detected by irradiating an occupant seated on a driver's seat with a detection wave, such as an electromagnetic wave, and by sensing the difference between the detection wave and a reflected wave thereof, and in which an irradiation range is caused to follow in accordance with a front-back movement of the driver's seat.

Japanese Unexamined Patent Application Publication No. 2019-199177 describes a technology in which, for example, a brain wave or a pulse of a driver is measured by using a contact sensor provided at a wearable terminal, a steering wheel, or the like, to determine whether a driver is capable of driving.

Japanese Unexamined Patent Application Publication No. 2019-34644 describes a technology in which, for example, a photoelectric pulse-wave sensor that measures a pulse wave of an occupant by using light, a piezoelectric pulse-wave sensor that measures a pressure wave at a surface of the body of an occupant, or an electromagnetic pulse-wave sensor that measures a pulse wave of an occupant by using an electromagnetic wave is provided inside a seat of a vehicle.

### SUMMARY

A vital sign measurement device according to an aspect of the disclosure includes at least one sensor that is provided at a seat, and that is configured to contact a finger of a measurement subject and to measure a vital sign of the measurement subject. The seat includes a seat surface portion on which a femoral region and buttocks of the measurement subject are to be placed and a seat back capable of being disposed along a back of the measurement subject. The at least one sensor includes at least one side-portion sensor that is disposed below a side edge of the seat surface portion.

A vital sign measurement device according to another aspect of the disclosure includes at least one sensor that is provided at a seat, and that is configured to contact a finger of a measurement subject and to measure a vital sign of the measurement subject. The seat includes a seat surface portion on which a femoral region and buttocks of the measurement subject are to be placed and a seat back capable of being disposed along a back of the measurement subject. The at least one sensor includes a front-portion sensor that is disposed below a region in proximity to a side end of a front edge of the seat surface portion.

### BRIEF DESCRIPTION OF THE DRAWINGS

The accompanying drawings are included to provide a further understanding of the disclosure and are incorporated in and constitute a part of this specification. The drawings illustrate an example embodiment and, together with the specification, serve to describe the principles of the disclosure.

FIG. 1 is a schematic external perspective view of a vehicle seat at which an embodiment of a vital sign measurement device to which the disclosure is applied is provided, and illustrates a state in which the upper body of an occupant is reclining on a seat back.

FIG. 2 is a schematic external perspective view of the vehicle seat at which the vital sign measurement device of the embodiment is provided, and illustrates a state in which the upper body of the occupant has fallen forward and an arm has descended on a side of a seat surface portion.

FIG. 3 is a schematic external perspective view of the vehicle seat at which the vital sign measurement device of the embodiment is provided, and illustrates a state in which the upper body of the occupant has fallen forward and the arm has descended forwardly of the seat surface portion.

FIG. 4 is a schematic external perspective view of the vehicle seat at which the vital sign measurement device of the embodiment is provided, and illustrates a state in which a lever of each sensor has been moved out.

FIG. 5 is a schematic plan view of a side portion of the seat surface portion with a grip portion of each sensor in a protruding state in the vehicle seat of the embodiment.

FIG. 6 is a schematic sectional view of a back side-portion sensor in the vital sign measurement device of the embodiment, and illustrates a normal state (state before a collision).

FIG. 7 is a schematic sectional view of the back side-portion sensor in the vital sign measurement device of the embodiment, and illustrates a state immediately after a collision.

FIG. 8 is a schematic sectional view of the back side-portion sensor in the vital sign measurement device of the embodiment, and illustrates a state when measuring vital signs.

### DETAILED DESCRIPTION

An effective method for grasping the condition of an occupant after a collision is, for example, measuring a vital sign, such as arterial oxygen saturation SpO₂ or a pulse rate.

However, when, for example, a vital sign is measured in a non-contact state by using a camera, after a collision, a frame-out of an occupant to the outside of an imaging angle of view of the camera may occur, or an extraneous material may be interposed between the camera and the occupant. Even when a vital sign is measured in a non-contact state by using a detection wave, such as an electromagnetic wave, the same problem may occur.

On the other hand, although a sensor for measuring a vital sign is also proposed as being built in a seat, in this case, a vital sign may not be capable of being precisely measured due to the clothes of an occupant.

In this respect, if, for example, a sensor that directly contacts the fingers or the like of an occupant is used, the precision of measurement of a vital sign is ensured. However, it may be difficult to cause an occupant who has been injured or who is panicking after a collision to make himself extend his hand to the sensor for measuring a vital sign.

It is desirable to provide a vital sign measurement device that easily measures a vital sign of a measurement subject after a collision.

In the following, an embodiment of the disclosure is described in detail with reference to the accompanying drawings. Note that the following description is directed to an illustrative example of the disclosure and not to be construed as limiting to the disclosure. Factors including, without limitation, numerical values, shapes, materials, components, positions of the components, and how the components are coupled to each other are illustrative only and not to be construed as limiting to the disclosure. Further, elements in the following example embodiment which are not recited in a most-generic independent claim of the disclosure are optional and may be provided on an as-needed basis. The drawings are schematic and are not intended to be drawn to scale. Throughout the present specification and the drawings, elements having substantially the same function and configuration are denoted with the same numerals to avoid any redundant description.

The vital sign measurement device of the embodiment is, for example, a device that is provided at a front seat (driver's seat, assistant driver's seat) of an automobile, such as a car, and that measures vital signs of an occupant (measurement subject) seated on the seat after a collision.

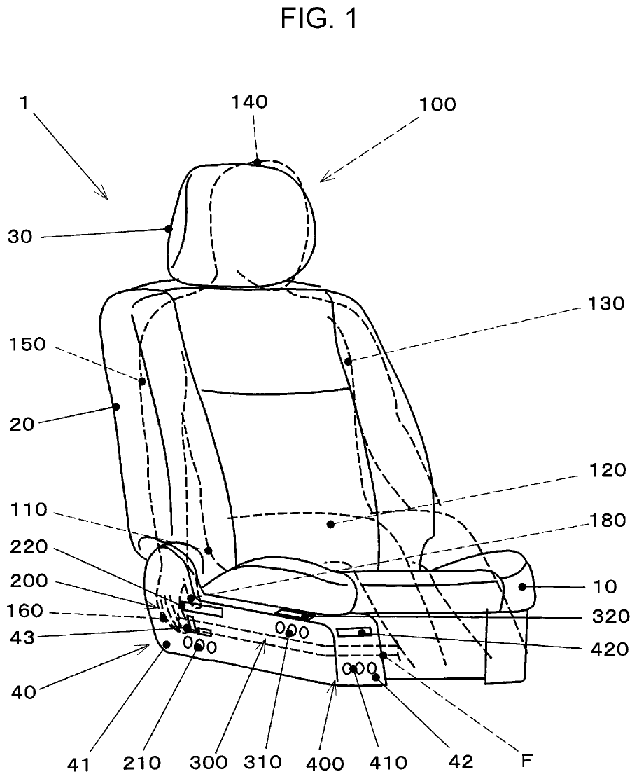

FIG. 1 is a schematic external perspective view of a vehicle seat at which the vital sign measurement device of the embodiment is provided, and illustrates a state in which the upper body of an occupant is reclining on a seat back.

A seat 1 includes, for example, a seat surface portion 10, a seat back 20, a headrest 30, and a seat trim 40.

The seat 1 is attached to a vehicle body (not illustrated) with an occupant 100 being oriented so as to face a forward direction of a vehicle.

The seat surface portion 10 is a portion on which buttocks 110 and a femoral region 120 of the occupant 100 are to be placed.

The seat surface portion 10 has a cushion placed on a seat frame F.

The seat back 20 is a backrest-like portion disposed along the back of an upper body 130 of the occupant 100.

The seat back 20 is formed so as to extend upward from the vicinity of a back end of the seat surface portion 10.

The headrest 30 is a portion that is provided so as to protrude upward from an upper end of the seat back 20 and that supports a back portion of a head 140 of the occupant 100.

The seat trim 40 is a cover-like member that is provided below the seat surface portion 10 and that covers, for example, the seat frame F, provided inside the seat 1, a seat rail (not illustrated), and a driving mechanism for changing a seat position.

The seat frame F is a frame-body-like structural member that is provided below the seat surface portion 10 and that is formed from, for example, a metallic member having a closed cross section.

The seat trim 40 has, for example, a side surface portion 41 and a front surface portion 42.

The side surface portion 41 is a surface portion formed by extending downward from a side edge of the seat surface portion 10.

The side surface portion 41 is disposed so as to face a lateral direction of the vehicle.

The front surface portion 42 is a surface portion formed by extending downward from a region in the vicinity of a side end of a front edge of the seat surface portion 10.

The front surface portion is disposed so as to face the forward direction of the vehicle.

A switch 43 for allowing the occupant 100 to change a seat position, such as a front position, a back position, an upward position, a downward position, an inclination in a forward direction, or an inclination in a backward direction of the seat surface portion 10, or to change an inclination (reclining angle) of the seat back 20 is provided at a back portion of the side surface portion 41.

The seat 1 is provided with, for example, sensors that measure vital signs of the occupant 100 after a collision.

As vital signs, for example, a pulse and an arterial oxygen saturation SpO₂ can be measured.

The seat 1 has, for example, a back side-portion sensor 200, a front side-portion sensor 300, and a front-portion sensor 400.

The back side-portion sensor 200 is provided at the back portion of the side surface portion 41 of the seat trim 40.

The back side-portion sensor 200 is typically a sensor that, when, after a collision, the upper body 130 of the occupant 100 is reclining on the seat back 20, measures vital signs of the occupant 100 whose arm 150 is in a descended state on a side of the seat surface portion 10.

The back side-portion sensor 200 has, for example, openings 210 and a lever 220.

The openings 210 are portions into which fingers 160 of the occupant 100 are inserted when measuring vital signs.

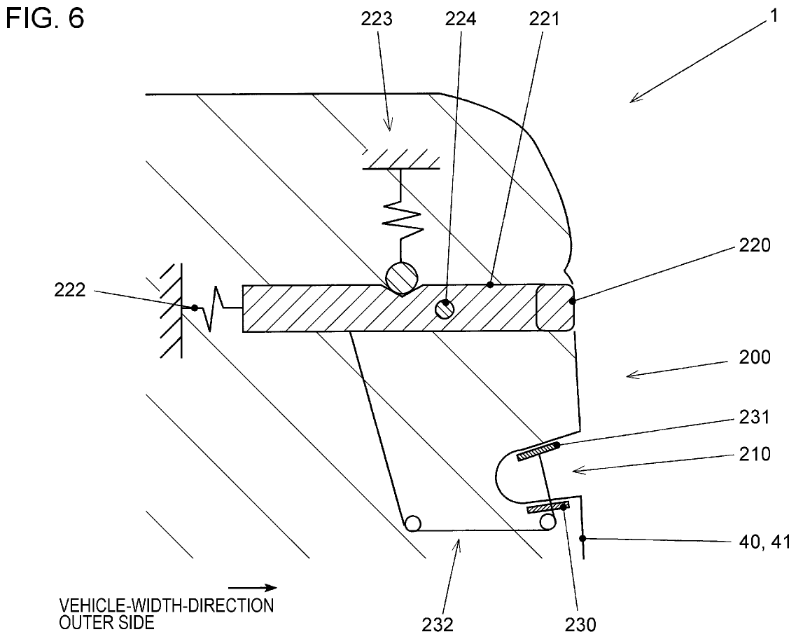

A probe 230 of a finger-tip pulse oximeter (see FIGS. 6 to 8), which is a detector of the back side-portion sensor 200, is provided inside the openings 210.

For example, there are three openings 210 for allowing the occupant 100 to insert the index finger, the middle finger, and the ring finger. (This applies to openings 310 and openings 410 described below.)

The openings 210 are disposed in, for example, a vehicle front-back direction.

The openings 210 are disposed below the switch 43.

The lever 220 is a member that guides the fingers 160 of the occupant 100 into the openings 210 in a collision.

The lever 220 is disposed above the openings 210 and the switch 43.

In general, in the human body, when, in a weakened state, the arms are descended on sides of the trunk of the body, the palms of the hands are often oriented inward (toward the trunk/buttocks).

Therefore, the longitudinal direction of the lever 220 is the vehicle front-back direction.

The lever 220 is accommodated in or is adjacent to the side surface portion 41 of the seat trim 40 in a normal state (before a collision), and is moved out outward in a vehicle-width direction so as to protrude from the side surface portion 41 after the collision.

The structure, operation, and function of the lever 220 are described in detail below.

The front side-portion sensor 300 is provided at a front portion of the side surface portion 41 of the seat trim 40.

The front side-portion sensor 300 is typically a sensor that, when, after a collision, the upper body 130 of the occupant 100 is in a forwardly fallen state, measures vital signs of the occupant 100 whose arm 150 is in a descended state on the side of the seat surface portion 10.

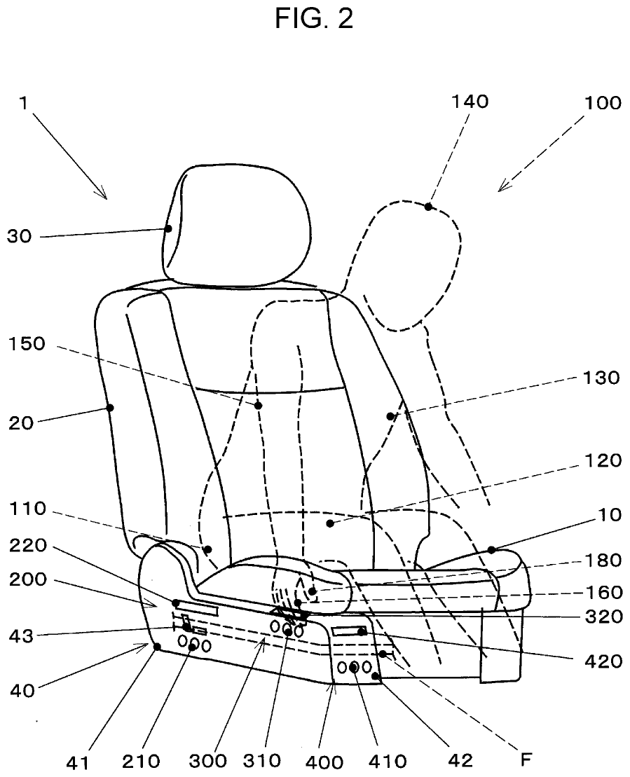

FIG. 2 is a schematic external perspective view of the vehicle seat at which the vital sign measurement device of the embodiment is provided, and illustrates a state in which the upper body of the occupant has fallen forward and the arm has descended on the side of the seat surface portion.

The front side-portion sensor 300 has, for example, the openings 310 and a lever 320.

The openings 310 are portions into which the fingers 160 of the occupant 100 are inserted when measuring vital signs.

A probe (not illustrated) of a finger-tip pulse oximeter, which is a detector of the front side-portion sensor 300, is provided inside the openings 310.

There are, for example, three openings 310 that are disposed in the vehicle front-back direction.

The openings 310 of the front side-portion sensor 300 are disposed at positions in an up-down direction that are higher than the positions of the openings 210 of the back side-portion sensor 200.

For example, although the openings 210 are disposed at positions that are lower than the position of the seat frame F, the openings 310 can be disposed at positions that are higher than the position of the seat frame F.

Such a difference between the height of the openings 210 and the height of the openings 310 is set by considering the difference between the height of the fingers 160 when the upper body 130 of the occupant 100 is reclining on the seat back 20 and the height of the fingers 160 when the upper body 130 of the occupant 100 is in a forwardly inclined state.

The lever 320 is a member that guides the fingers 160 of the occupant 100 into the openings 310 in a collision.

The lever 320 is disposed above the openings 310 and near an upper edge of the side surface portion 41 of the seat trim 40.

The longitudinal direction of the lever 320 is the vehicle front-back direction.

The lever 320 is accommodated in or is adjacent to the side surface portion 41 in a normal state, and is moved out upward so as to protrude from the side surface portion 41 after a collision.

The front-portion sensor 400 is provided at a front portion of the seat trim 40.

The front-portion sensor 400 is typically a sensor that, when, after a collision, the upper body 130 of the occupant 100 is in a forwardly fallen state, measures vital signs of the occupant 100 whose arm 150 is in a descended state along an outer side of the femoral region 120 forwardly of the seat surface portion 10.

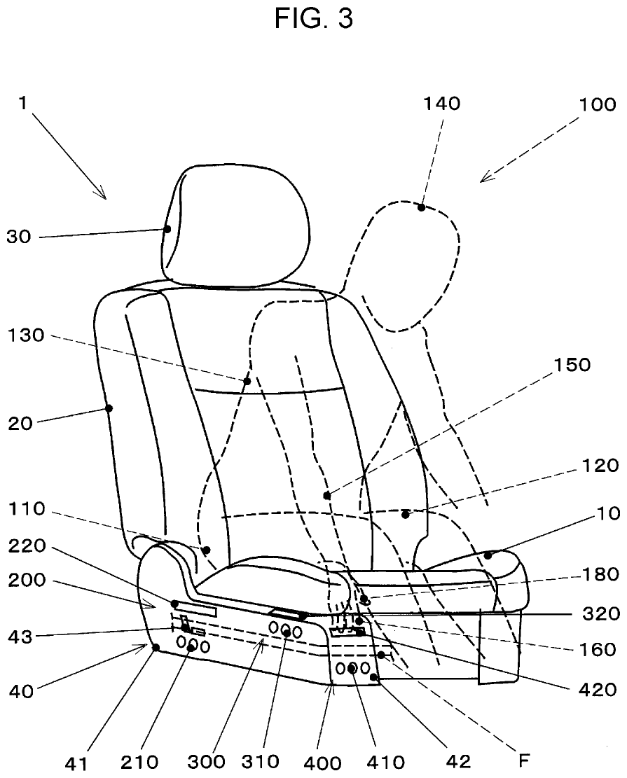

FIG. 3 is a schematic external perspective view of a side portion of the seat surface portion with a grip of each sensor in a protruding state in the vehicle seat of the embodiment.

The front-portion sensor 400 has, for example, the openings 410 and a lever 420.

The openings 410 are portions into which the fingers 160 of the occupant 100 are inserted when measuring vital signs.

A probe (not illustrated) of a finger-tip pulse oximeter, which is a detector of the front-portion sensor 400, is provided inside the openings 410.

There are, for example, three openings 410 that are disposed in the vehicle-width direction.

The lever 420 is a member that guides the fingers 160 of the occupant 100 into the openings 410 in a collision.

The lever 420 is disposed above the openings 410 and near an upper edge of the front surface portion 42 of the seat trim 40.

In general, in the human body, when, in a weakened state, the arms have descended forwardly of the trunk of the body, the backs of the hands are often oriented in the forward direction.

Therefore, the longitudinal direction of the lever 420 is the vehicle-width direction.

The lever 420 is accommodated in or is adjacent to the front surface portion 42 in a normal state, and is moved out forwardly of the vehicle so as to protrude from the front surface portion 42 after a collision.

The levers 220, 320, and 420 described above are moved out so as to protrude from the seat 1 in response to the acceleration to which a vehicle body is subjected in a collision.

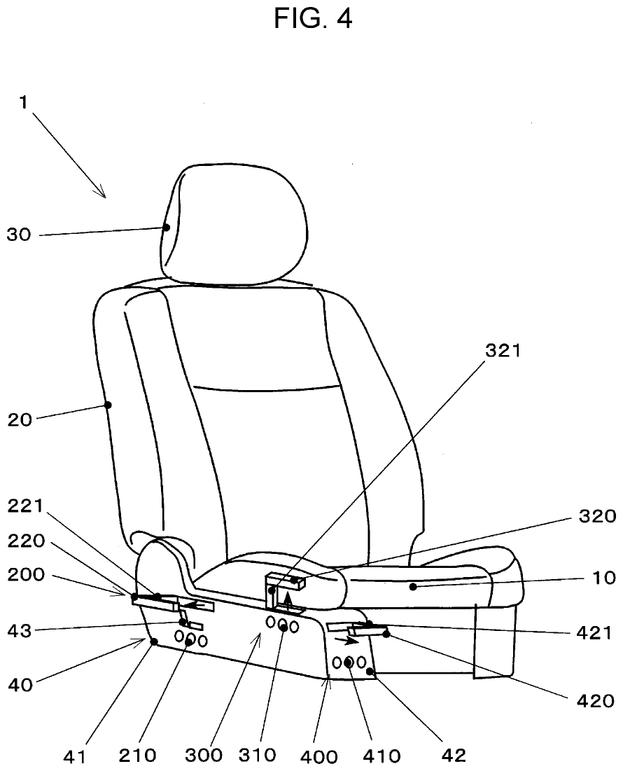

FIG. 4 is a schematic external perspective view of the vehicle seat at which the vital sign measurement device of the embodiment is provided, and illustrates a state in which the lever of each sensor has been moved out.

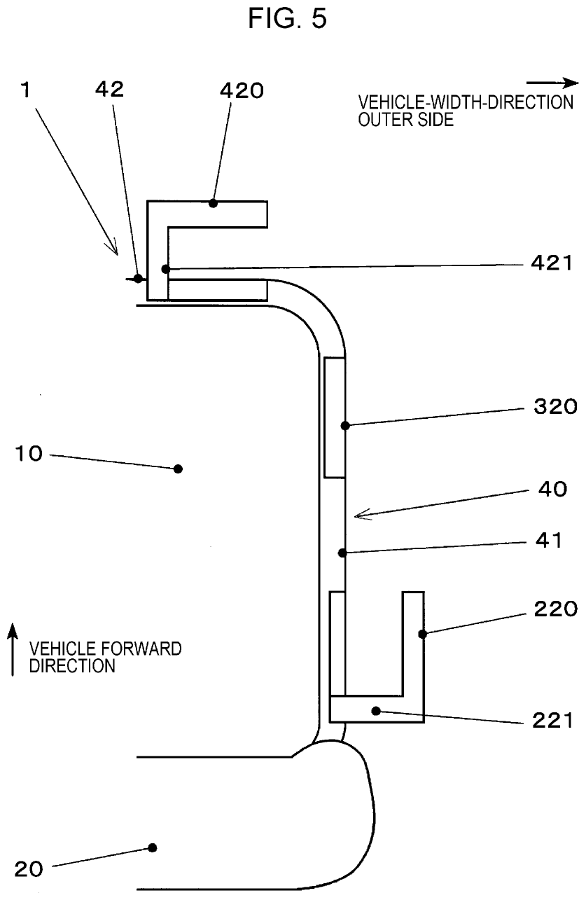

FIG. 5 is a schematic plan view of the side portion of the seat surface portion with the grip of each sensor in a protruding state in the vehicle seat of the embodiment.

The lever 220 of the back side-portion sensor 200 is moved out outward in the vehicle-width direction from the side surface portion 41 of the seat trim 40.

In the moved-out state, the lever 220 is supported by a stay 221.

The stay 221 is provided so as to protrude inward in the vehicle-width direction from an end of the lever 220 situated toward a back side of the vehicle.

Although a gap into which a thumb of the occupant 100 is inserted is formed between the lever 220 and the side surface portion 41 of the seat trim 40, the thumb can be inserted into the gap from either one of an upper side and a forward side of the vehicle.

The lever 320 of the front side-portion sensor 300 is moved out upward from the side surface portion 41 of the seat trim 40.

In the moved-out state, the lever 320 is supported by a stay 321.

The stay 321 protrudes downward from an end of the lever 320 situated toward the back side of the vehicle.

The lever 320 is disposed so as to be interposed between the fingers 160 and the thumb of the occupant 100 when the arm 150 has descended in a state in which the upper body 130 of the occupant 100 has fallen forward.

The lever 420 of the front-portion sensor 400 is moved out in the forward direction of the vehicle from the front surface portion 42 of the seat trim 40.

In the moved-out state, the lever 420 is supported by a stay 421.

The stay 421 protrudes backward from an end on an inner side in the vehicle-width direction of the lever 420.

Although a gap into which a thumb of the occupant 100 is inserted is formed between the lever 420 and the front surface portion 42 of the seat trim 40, the thumb can be inserted into the gap from either one of the upper side and an outer side in the vehicle-width direction.

The functions of the levers 220, 320, and 420 described above of guiding the fingers 160 of the occupant 100 toward a measurement position that contacts the probe are described by taking as an example the lever 220 of the back side-portion sensor 200.

Note that the levers 320 and 420 are substantially the same in terms of operations and effects, and differ in the protruding direction from the seat 1.

FIG. 6 is a schematic sectional view of the back side-portion sensor in the vital sign measurement device of the embodiment, and illustrates a normal state (state before a collision).

The back side-portion sensor 200 further includes a spring 222, a lock mechanism 223, a support shaft 224, the probe 230, a plate 231, and an interlocking mechanism 232.

The spring 222 is a spring that is provided on an end on a side opposite to a lever-220 side of the stay 221, and that urges and pushes the stay 221 in a move-out direction of the lever 220 (outer side in the vehicle-width direction in the case of the lever 220).

The lock mechanism 223 is a mechanism that is formed by pushing a weight into a recess in the stay 221 by an urging member, such as a spring; and that, during normal use of a vehicle (other than in a collision), locks the stay 221 and prevents the lever 220 from being moved out by the spring 222, and that, in a collision, unlocks the stay 221 and allows the lever 220 to be moved out by the spring 222.

The lock mechanism 223 is formed so as to be unlocked when an acceleration of a magnitude that is not produced during normal use of a vehicle (for example, approximately 10 G) is produced in the vehicle body.

The support shaft 224 is a rotation center shaft that, in a state in which the lever 220 is moved out outward in the vehicle-width direction and protrudes from the side surface portion 41 of the seat trim 40, rotatably (swingably) supports the stay 221 in a direction in which the lever 220 is moved downward.

The probe 230 is a detector that measures vital signs from the fingers 160 of the occupant 100.

The probe 230 has, for example, the function of detecting the arterial oxygen saturation SpO₂, the function of obtaining a pulse rate, or the like by irradiating the nails of the fingers 160 of the occupant 100 with light that a red LED emits.

The probe 230 is provided at a lower surface portion of the openings 210.

The plate 231 is a pressurizing member that causes the nails to press-contact the probe 230 by coming into contact with a finger-print side (upper side) of the fingers 160 of the occupant 100 and being pushed downward.

The plate 231 is adjacent to an upper surface defining the openings 210 and is attached to the openings 210 so as to be displaceable relative to the openings 210 in the up-down direction.

The interlocking mechanism 232 is a mechanism that operates in accordance with the rotation of the stay 221 around the support shaft 224, and that moves the plate 231 downward.

Although, as the interlocking mechanism 232, it is possible to use, for example, a structure in which a region of the stay 221 that is situated inward of the support shaft 224 in the vehicle-width direction is coupled to a lower surface of the plate 231 with a cord-like member having flexibility and elasticity and in which the plate 231 is moved downward in accordance with the rotation of the stay 221, the structure of the interlocking mechanism 232 is not limited thereto and can be changed as appropriate.

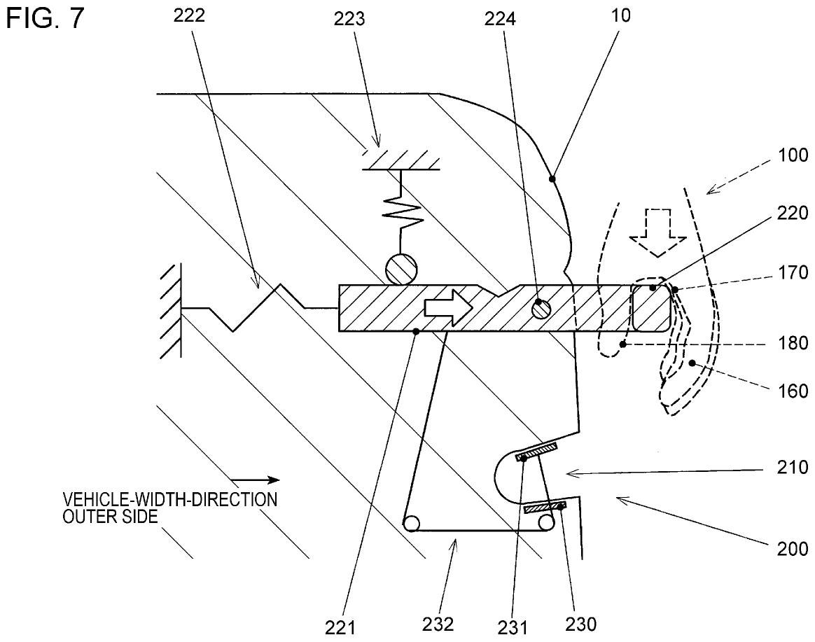

FIG. 7 is a schematic sectional view of the back side-portion sensor in the vital sign measurement device of the embodiment, and illustrates a state immediately after a collision.

After a collision, the lock mechanism 223 is unlocked in accordance with the acceleration to which the vehicle body is subjected, and the lever 220 and the stay 221 are moved out in the vehicle-width direction by the spring 222.

Therefore, the lever 220 is in a protruded state from the seat 1 with the gap between the lever 220 and the side surface portion 41 of the seat trim 40 being provided.

When the arm 150 has been moved downward due to its own weight in a state in which, due to a shock of a collision, the occupant 100 has his consciousness dimmed and has his body weakened and thus has his upper body 130 reclining on the seat back 20, the lever 220 is interposed between a thumb 180 and the fingers 160 and a palm 170.

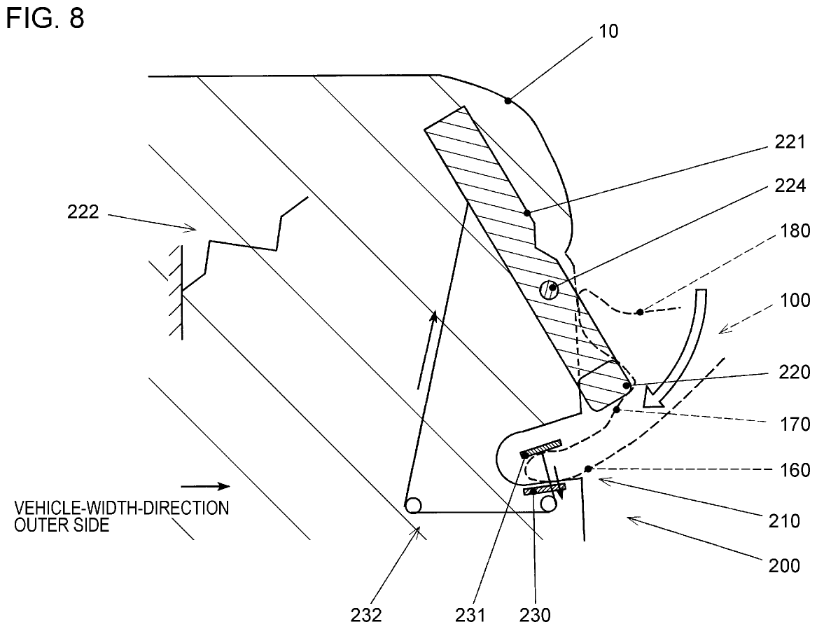

FIG. 8 is a schematic sectional view of the back side-portion sensor in the vital sign measurement device of the embodiment, and illustrates a state when measuring vital signs.

The lever 220 rotates in a direction in which the lever 220 moves downward around the support shaft 224 from the state illustrated in FIG. 7 due to the self-weight of the arm 150 of the occupant 100.

At this time, the spring 222 and the lock mechanism 223 can be formed to engage with and disengage from the stay 221 so as not to interfere with the rotation of the stay 221.

The fingers 160 of the occupant 100 follow the rotation of the lever 220 and rotate around the support shaft 224, and the fingertips are guided so as to be inserted into and accommodated in the openings 210.

Note that, since, when the occupant 100 is in a weakened state, the joints of the fingers 160 are ordinarily often in a gently bent state, the fingertips can be inserted into the openings 210 by making use of the bending of the joints.

The interlocking mechanism 232 moves the plate 231 downward in accordance with the rotation of the stay 221 around the support shaft 224.

Therefore, the fingertips of the fingers 160 of the occupant 100 are pushed and restrained so that the nails press-contact the probe 230.

In accordance with the ending of the rotation of the stay 221 (ending of the insertion of the fingers 160 into the openings 210), the probe 230 starts to measure vital signs, such as the arterial oxygen saturation SpO₂ and a pulse rate, of the occupant.

Note that the measured vital signs can be automatically transmitted to a remote base station (not illustrated) via a communication device (not illustrated).

The base station can share the vital signs of the occupant obtained from a vehicle in a collision with, for example, an organization, such as an emergency organization, a medical organization, or a police organization.

Similarly to the lever 220 described above, the lever 320 of the front side-portion sensor 300 and the lever 420 of the front-portion sensor 400 are each moved out so as to protrude from the seat 1 in a collision and each have the function of guiding the fingers of the occupant 100 so as to be inserted into the openings 310 or 410.

As described above, the present embodiment can provide the following effects.

(1) By providing the back side-portion sensor 200 and the front side-portion sensor 300 below a side edge of the seat surface portion 10, even if, due to a shock of a collision or the like, the occupant 100, who is a measurement subject, has his consciousness dimmed and is in a weakened state, the fingers 160 naturally easily reach each sensor with the arm 150 being unrestrained and hanging loosely on the side of the seat 1, and vital signs of the occupant 100 can be easily measured after the collision.
(2) In a state in which the occupant 100 has his consciousness dimmed after a collision, the upper body 130 of the occupant 100 is in a forwardly fallen state or in a reclining state on the seat back 20.

By disposing the back side-portion sensor 200 and the front side-portion sensor 300 apart from each other in the front-back direction, since, in a state in which the upper body 130 of the occupant 100 has fallen forward, the arm 150 descends to the side on a forward side of the seat surface portion 10 of the seat 1, vital signs can be measured by the front side-portion sensor 300.

On the other hand, since, in a state in which the upper body 130 of the occupant 100 reclines on the seat back 20, the arm 150 descends to a side on a backward side of the seat surface portion 10 of the seat 1, vital signs can be measured by the back side-portion sensor 200.

(3) In a state in which the upper body 130 of the occupant 100 reclines on the seat back 20, the positions of the hands when the arm 150 has descended are often lower than when the upper body 130 is in the forwardly fallen state. In accordance with such a difference in height according to the posture of the upper body 130, vital signs can be easily measured for each posture by disposing the front side-portion sensor 300 and the back side-portion sensor 200 at different heights.
(4) By providing the front-portion sensor 400 that is disposed below a region in proximity to a side end of the front edge of the seat surface portion 10, in a state in which the upper body 130 of the occupant 100 has fallen forward and the arm 150 has descended forwardly of the seat surface portion 10 of the seat 1, the fingers 160 naturally easily reach the front-portion sensor 400, and vital signs of the occupant 100 can be easily measured after a collision.

Note that it is confirmed that the installation positions of the sensors of the present embodiment described above are positions where each sensor functions effectively (where the fingers come close to the sensors in a state in which the occupant has been weakened and the arm has descended) even if the body size of a measurement subject changes, for example, from 165 cm to 190 cm in height.

As described above, according to the disclosure, a vital sign measurement device that easily measures a vital sign of a measurement subject after a collision can be provided.

### Modifications

The disclosure is not limited to the embodiment described above, and various modifications and changes can be made, and such modifications and changes are also within the technical scope of the disclosure.

(1) For example, the shapes, the structures, the dispositions, the materials, the manufacturing methods, and the number of members of the vital sign measurement device and the seat are not limited to those of the embodiment described above, and can be changed as appropriate.
(2) The structures of, for example, the sensors that measure vital signs in the embodiment and the mechanisms that guide the fingers of a measurement subject to the sensors are each one example, and can be changed as appropriate.

Although, in the embodiment, as vital signs, an arterial oxygen saturation concentration and a pulse rate are measured, the disclosure may be such that one of the arterial oxygen saturation concentration and the pulse rate is measured. Other vital signs may be additionally or selectively measured. For example, the blood pressure may be measured.

(3) Although, in the embodiment, as sensors that measure vital signs, the back side-portion sensor, the front side-portion sensor, and the front-portion sensor are provided, one or two of these sensors may be provided. A sensor or sensors other than these sensors may be additionally provided.
(4) The structures of the mechanisms that guide the fingers of a measurement subject to measurement positions are not limited to the structures of the embodiment, and can be changed as appropriate. For example, the shapes of the levers, the method of moving out the levers, the structures of the mechanisms that move out the levers, and the movement paths of the levers when guiding the fingers can be changed as appropriate.

Although, in the embodiment, each lever is formed so as to be moved out by a spring in accordance with a shock of a collision, each lever is not limited thereto, and, for example, each lever may be moved out by using various actuators, such as an electrical actuator. For example, when a pre-crash has been determined based on a sensor output of a stereo camera device, a millimeter wave radar device, or the like (when there is a sign of a collision), the levers may be moved out before the actual collision.

Further, each lever may protrude from the seat during normal use of a vehicle. In this case, a mechanism for moving out each lever is not used, and the structure of the device can be simplified.

(5) Although, in the embodiment, the seat is provided as, for example, a front seat of an automobile, such as a car, the type of vehicle and the installation location of the seat are not limited thereto.

For example, the seat may be installed in a second row or a subsequent row of a car.

1. A vital sign measurement device comprising:
at least one sensor that is provided at a seat, and that is configured to contact a finger of a measurement subject and to measure a vital sign of the measurement subject, the seat comprising a seat surface portion on which a femoral region and buttocks of the measurement subject are to be placed and a seat back capable of being disposed along a back of the measurement subject,
wherein the at least one sensor includes at least one side-portion sensor that is disposed below a side edge of the seat surface portion.

2. The vital sign measurement device according to claim 1, wherein the at least one side-portion sensor includes a front side-portion sensor and a back side-portion sensor that are disposed apart from each other in a front-back direction of the seat surface portion.

3. The vital sign measurement device according to claim 2, wherein the back side-portion sensor is disposed at a position that is lower than a position of the front side-portion sensor.

4. The vital sign measurement device according to claim 1, wherein the at least one sensor includes a front-portion sensor that is disposed below a region in proximity to a side end of a front edge of the seat surface portion.

5. The vital sign measurement device according to claim 2, wherein the at least one sensor includes a front-portion sensor that is disposed below a region in proximity to a side end of a front edge of the seat surface portion.

6. The vital sign measurement device according to claim 3, wherein the at least one sensor includes a front-portion sensor that is disposed below a region in proximity to a side end of a front edge of the seat surface portion.

7. A vital sign measurement device comprising:
at least one sensor that is provided at a seat, and that is configured to contact a finger of a measurement subject and to measure a vital sign of the measurement subject, the seat comprising a seat surface portion on which a femoral region and buttocks of the measurement subject are to be placed and a seat back capable of being disposed along a back of the measurement subject,
wherein the at least one sensor includes a front-portion sensor that is disposed below a region in proximity to a side end of a front edge of the seat surface portion.
